(19) United States
(12) Patent Application Publication
(10) Pub. No.: US 2024/0088727 A1
(43) Pub. Date: Mar. 14, 2024

(54) CHARGING DEVICE

(71) Applicants: Walter MEHNERT, Ottobrunn (DE); Thomas THEIL, Feldafing (DE)

(72) Inventors: Walter MEHNERT, Ottobrunn (DE); Thomas THEIL, Feldafing (DE)

(21) Appl. No.: 18/275,049

(22) PCT Filed: Feb. 1, 2022
(86) PCT No.: PCT/EP2022/052313

§ 371 Date: Jul. 31, 2023

(30) Foreign Application Priority Data
Feb. 1, 2021 (DE) 10 2021 102 244.4
Apr. 16, 2021 (DE) 10 2021 109 685.5
Aug. 25, 2021 (EP) 21193145.6

(51) Int. Cl. H02J 50/90; H02J 50/00; H02J 50/12

(52) U.S. Cl. CPC H02J 50/90; H02J 50/005; H02J 50/12; A61N 1/3787; H02J 2310/23

(57) ABSTRACT

The invention relates to a charging device (100, 200, 300) for contactlessly charging an energy storage of an implant (I) implanted in a body of a living being, comprising:
at least one self-supporting coil (110, 210) extending along a coil axis and configured to generate alternating magnetic field; wherein
during intended use of the charging device (100, 200, 300), the body is arranged relative to the coil (110, 210) so that the alternating magnetic field extending in the area inside the coil along the coil axis penetrates the body for charging the energy storage.

The present invention relates to a charging device for contactless charging of an energy storage of an implant.

An electronic pacemaker to be fully implanted into a body of a living being is known from the patent document DE 10 2018 205 940 A1. This pacemaker comprises an energy storage that supplies electric energy to an electronics assembly of the pacemaker and that, after discharge, can be contactlessly charged.

The contactless recharging of the energy storage takes place via “indirect” induction with the help of a magnetization portion which responds to an alternating magnetic field in such a way that a wave of reversal domains (Weiss domains) occurs in it when a certain magnetic field strength is reached. The occurrence of the wave is independent on the frequency of the alternating magnetic field, which only provides information about how often the wave per unit of time occurs.

Because of the quick change of the magnetic flux, this wave causes a coil arranged in the spatial proximity to emit a voltage impulse leading to the charging of the energy storage.

A charging device shown in the patent document generates the alternating magnetic field, wherein the charging device is arranged on or near a surface of the body of the living being during the charging process, and the alternating magnetic field is to trigger the magnetization portion of the pacemaker in the manner explained.

The charging device comprises a plurality of coils for the generation of the alternating magnetic field. The area of the alternating magnetic field that penetrates the surface of the body and reaches the magnetization portion is part of the weak stray field of the coils.

This stray field of the coils is at the same time extremely inhomogeneous and strongly decreases with the increasing distance from the coil.

For this reason, the recharging of the energy storage requires, depending on the implantation depth of the pacemaker, a meticulous adjustment of the electrical operating parameters of said coils and a strong stray field with the corresponding direction of the field vector, so that the wave of reversal domains in the magnetization portion is generated reliably and periodically.

If the magnetic field in the area of the magnetization portion deviates too much from a certain optimal value of the field strength or flux density, either because it is too weak or too inhomogeneous, the wave that is required for charging will not be triggered.

In order to achieve the necessary field strength or flux density in the stray field, the known charging device must have a very high current/voltage resistant design.

Attempting to charge the energy store by means of ordinary (frequency-dependent) induction according to the transformer principle, one faces, in addition to the problems explained, in particular with regard to field strength/flux density, even greater negative effects.

With this type of charging, frequencies which are as high as possible would be desirable in order to achieve adequate energy transmission with simultaneously acceptable field strength/flux density. With an increase in the frequency, however, adverse skin effects in the conductive human tissue of the body, at the same time, increase in such a way that the magnetic alternating field penetrates only slightly into the body and a reaching of the implant is impossible.

An increase in the field strength/flux density of the stray field at the location of the implant with at the same time a low frequency is alternatively also hardly possible, because inductive alternating-current-resistances of coils located in the charging device had to be selected such large that current and/or voltage values were inside ranges that are no longer practicable.

Against the above background, it is the object of the invention to provide a charging device for contactless charging of an energy storage of an implant, which allows an easier and more reliable generation of an alternating magnetic field necessary for charging.

Furthermore, it is the object of the invention to provide a simple charging device in regard to the prior art, in particular as far as the current and voltage values are concerned.

This/These object(s) is/are achieved by a charging device according to claim 1. Preferred embodiments of the charging device according to the invention are subject-matters of the dependent patent claims.

According to one aspect of the invention, the charging device for contactless charging of an energy storage of an implant implanted in a body of a living being includes the following features:
at least one self-supporting coil extending along a coil axis and configured to generate alternating magnetic field; wherein
during intended use of the charging device, the body is arranged relative to the coil so that the alternating magnetic field extending in the area inside the coil along the coil axis penetrates the body for charging the energy storage.

Strictly speaking, the coil of the charging device generates an electromagnetic alternating field. Nevertheless, what is of interest to the invention is only the magnetic portion of the electromagnetic alternating field. Therefore, the present description talks solely of an alternating magnetic field.

The implant whose energy storage is charged by the charging device according to the invention in accordance with the intended use is, for example, a cardiac pacemaker, a brain pacemaker, an organ pacemaker or an analysis unit. The last analysis unit is formed, for example, in such a way that it determines parameters, such as blood pressure and/or blood values, continuously or at specific time intervals. Particularly preferred is that the implant is a cardiac pacemaker or cardiac pacemaker network seated/implanted in or on the human heart.

In order to become independent of the value of the frequency of the alternating magnetic field, an essential element of the implant is a magnetization portion with oriented magnetic domains, across which runs a remagnetization wave in the form of the continuous reversal magnetic domains when the amplitude (B-field) of the alternating magnetic field generated by the charging device in the area of the magnetization portion reaches a certain value. The magnetization portion is, for example, an impulse wire or a Wiegand wire.

A coil located spatially close to the magnetization portion generates a voltage impulse due to the remagnetization wave and the corresponding quick change in magnetic flux, which supplies energy to charging electronics, for example, and leads to a charging impulse. The energy storage can be charged with the charging impulse. The alternating magnetic field generated by the charging device initiates the remagnetization wave per commutation, wherein charging impulses occur in the same number and charge the energy storage.

Alternatively, the implant may be designed differently, in that its energy storage is charged using ordinary frequency-depending induction. For this purpose, the implant has a receiving coil with a preferably specially shaped core, wherein the receiving coil emits charging impulses which are dependent on the frequency and magnitude of the alternating field generated. The receiving coil is wound around the core in the direction of a longitudinal extension.

The charging device according to the invention is therefore intended for charging utilizing “ordinary” frequency-depending induction and/or “indirect” frequency-independing induction.

A cardiac pacemaker or a cardiac pacemaker network—using “indirect” induction for charging—which comprise the magnetization portion explained hereinabove and the energy storage of which is charged by generating the remagnetization wave, are known from the patent documents DE 10 2019 124 435 and EP 3 756 726 A2.

The explanations contained in these patent documents with respect to the respective charging impulse generating portions, in particular with respect to the magnetizing portions including the coils wound therearound and generating the respective voltage impulse, and with respect to the respective charging electronics are incorporated by reference.

A frequency f of the alternating magnetic field generated by the charging device according to the invention is in particular between 0.1 kHz≤f≤10 kHz, in particular at f=2 kHz, 3 kHz, 4 kHz, or 5 kHz. The frequency f is preferably adapted to the spatial dimensions and material data of the magnetization portion and a related runtime of the remagnetization wave running across the magnetization portion.

The frequencies are also suitable for charging the implant in its alternative embodiment based on the ordinary induction, as far as the receiving coil comprises the specially shaped core.

The shape of the self-supporting coil of the charging device according to the invention can be differently designed. For example, the coil can be circular, rectangular or square.

The coil is designed in such a way that it is dimensionally-stable self-supporting, regardless of the body. This means that the body can move relative to the dimensionally-stably supported coil. In addition, the coil has such spatial dimensions that the body of the living being can be moved relative to the coil without any contact with the coil or its elements, such as frames or insulations, so that the body is ultimately located completely without contact with the coil and the implant is located in the area inside the coil and in the alternating magnetic field extending there along the coil axis, respectively.

The coil of the charging device according to the invention generates an almost homogeneous alternating magnetic field in the area extending inside the coil along the coil axis, wherein an amplitude of the corresponding magnetic flux density in an entire radially inner area of the coil with respect to the coil axis has such values that
i.—assuming the same orientation between the magnetic field (direction of the B-vector) and the intended running direction of the remagnetization wave—the remagnetization wave is initiated per commutation of the alternating magnetic field and thus the charging impulse is generated, or
ii.—assuming the same orientation between the magnetic field (direction of the B-vector) and the intended orientation of the core of the receiving coil, which core serving for the ordinary induction—the charging impulse is generated pro commutation of the alternating magnetic field.

Due to this field distribution, the exact positioning of the implant in the area inside the coil is not important, which makes a meticulous adjustment of the alternating field to the conditions of charging, such as depth of the implant, superfluous.

Preferably, the charging device is arranged to rotate a vector of the alternating magnetic field for charging at least spatially two-dimensional or spatially three-dimensional. In this respect, the charging device is preferably arranged to rotate the vector of the alternating magnetic field for charging without changing the corresponding amplitude of the vector. The rotation takes place mechanically, as will be described below with reference to a suspension, for example, or by changing superimposed individual alternating magnetic fields, which are located, for example, between sub-coils described below.

It is preferred that, during the intended use of the charging device, the body is arranged (contactless) relative to the self-supporting coil in such a way that a longitudinal axis of the body runs in the direction of the coil axis and is located within the coil, wherein the alternating magnetic field reaches the location of the implant.

In this preferred embodiment of the charging device, the longitudinal body axis of the living being, in particular of the human being, consequently runs in the direction of the coil axis, this embodiment being advantageous in particular if the orientation of the intended running direction of the remagnetization wave running across the magnetization portion or the orientation of the core serving for the ordinary induction coincides with the direction of the longitudinal body axis. This state can preferably be achieved by constructing the implant in such a way that the shape or the anchorages of the implant lead to this orientation.

Preferably, the charging device according to the invention further comprises:
an mount supporting the coil and configured to pivot the coil relative to the body about at least one, preferably two, axes, in order to rotate the vector, wherein the charging device is preferably configured, for example by a control unit, to drive the mount to pivot the coil to a particular orientation relative to the body to optimize charging of the energy storage.

Alternatively, the charging device can preferably be configured to actuate a display device in order to display an operator in which direction he/she is intended to pivot the coil manually relative to the body, in order to optimize the charging of the energy storage.

If the intended running direction of the remagnetization wave or the orientation of the core serving for ordinary induction does not coincide with the direction of the vector of the alternating magnetic field, the charging impulse may drop if the deviation is too great because the component of the magnetic field in the direction of the axis of the magnetization portion or the core becomes too small.

To allow adjustment or adaptation of the running direction of the remagnetization wave or core to the field orientation or vice versa, the mount is provided. The mount is, for example, a gimballed mount that allows the control unit of the charging device to pivot the coil of the charging device relative to the body of the living being about two axes to achieve the matching of the orientation (vector of the B-field) of the alternating magnetic field generated by the coil to the intended running direction of the remagnetization wave or core.

Advantageously, pivoting the coil does not entail any change in the amplitude of the magnetic flux density, but only changes the orientation of the alternating magnetic field.

Further, it is preferred that, the mount supporting the self-supporting coil is displaceable relative to the body and/or a body support for supporting the body is displaceable relative to the coil, wherein
the charging device is adapted to displace the mount and/or the body support to bring the self-supporting coil into a specific position relative to the body for optimizing the charging of the energy storage.

For example, the charging device includes a linear guide over which the entire mount of the coil is displaceable, preferably in the direction of the longitudinal axis of the body. Moving the mount along the linear guide can be performed by a user either manually or automatically by giving appropriate instructions at a user interface of the control unit. The displacement of the self-supporting coil (and/or the mentioned pivoting) takes place in such a way that the body of the living being, in particular of the human, contacts apart from the body support no other elements of the charging device, such as, for example, the coil or its elements.

The charging device is preferably configured to check whether or to what extent the orientation of the running direction of the remagnetizing wave or of the core serving for the ordinary induction corresponds to the orientation of the magnetic field. For example, the charging device is able to wirelessly request the state of charge of the energy storage at specific time intervals and draw a conclusion about the efficiency of charging based on the change in the state of charge of the energy storage, based on the commutation frequency and a known maximum level of charging impulses.

It is preferred, however, that the charging device comprises a receiving unit configured to receive a quality signal transmitted by the implant and reflecting the efficiency of the charging, and the charging device is configured, in dependence on the quality signal, to pivot and/or position the coil in the specific orientation to optimize the charging.

Said quality signal may, for example, be a low-frequency signal that penetrates the body of the living being and, if no respective antenna is provided, the sleeve or the case of the cardiac pacemaker. The quality of the charging impulse is preferably proportional to the value of the integral (∫idt). At good charging impulses, i.e. with very high quality, the value amounts up to 1000 nC, for example. The signal indicating the quality may, for example, indicate the value of the integral of the current of the charging impulse over time (∫idt), i.e. its charge content. Alternatively, the quality signal can indicate the following ratio:
(charge content of the delivered charging impulse/maximum possible charge content).

Further alternatively, the quality signal may be a binary signal that assumes an OK state when the charging impulse exceeds a certain threshold.

While observing the quality signal, the control unit controls the mount, for example the gimballed mount, and determines an optimal position of the coil from various positions of the coil and the corresponding quality signals, into which position the control unit finally pivots the coil. In the explained alternative, the charging device can display directions on the display device in which the operator is to pivot the coil.

The following are preferred parameters of the charging device and/or its coil according to the invention:

The magnetic flux density of the alternating magnetic field along the coil axis preferably has a value B in the mT range, preferably greater than/equal 1 mT, in particular in the following ranges:
1.0 mT<=B<=20.0 mT, especially 2.0 mT<=8<=20.0 mT, 2.5 mT<=8<=8.0 mT, 3.5 mT<=8<=7.0 mT, 4.5 mT<=8<=6.0 mT, 4.8 mT<=8<=5.2 mT, or 5.0 mT=8.

A diameter of the coil has a value D which is dimensioned in such a way that the body of the living being, in particular of the human, can be brought into the magnetic alternating field without coming into contact with the coil or its elements, which field extends in the region within the coil along the coil axis, and is arranged there, as intended for charging, in a contactless manner relative to the coil. The diameter is preferably in the following ranges: 0.6 m<=D<=0.9 m, 0.65 m<=D<=0.85 m, 0.68 m<=D<=0.8 m, or 0.72 m=D; and/or
a length of the coil preferably has a value I, where preferably 0.15 m<=I<=0.5 m, 0.2 m<=I<=0.45 m, 0.25 m<=I<=0.4 m, or 0.33 m=I.

The coil of the charging device may be a continuously wound coil, especially in the case where the longitudinal body axis of the human body is intended to coincide with the coil axis.

Alternatively, the coil can be pulled apart and build from two sub-coils. The dimensions of the sub-coils and their spacing from each other are preferably selected so that the coil pulled apart acts like a continuously wound coil, at least in a radial area around the coil axis. An example of a coil pulled apart is a Helmholtz coil. In accordance with the intended use, the human body is located between the sub-coils and the magnetic field and magnetic alternating field, respectively, with the longitudinal axis of the body running perpendicular to the coil axis.

The coil pulled apart can also be supported by an adjustable mount.

Further alternatively, the charging device may include two or three pulled apart coils with the coil axes preferably arranged in the two or three spatial coordinates. The human body and the implant are located between the respective sub-coils when the charging device is used as intended, wherein the alternating magnetic fields generated by the coils pulled apart, each in an area within the respective coil, are superimposed between the sub-coils and reach the implant implanted in the body.

Targeted orientation of the alternating magnetic field resulting from the superimposed alternating fields can be achieved by controlling the phases and/or the amplitudes of the respective pairs of coils differently, in order to optimize charging.

The control of the resulting alternating field can be, for example, based on the quality signal.

As can be seen from the second preferred embodiment and the third preferred embodiment of the charging device described in the following, the pulled apart coils mentioned above are self-supporting, i.e. the respective sub-coils arranged at a distance from one another are dimensionally stable and self-supporting, so that the body of the living being (humans) can be moved into the magnetic alternating field and stays there for charging contactless with the coil and its elements, respectively.

Hereinbelow, preferred embodiments of the charging device according to the invention will be explained with reference to the accompanying figures.

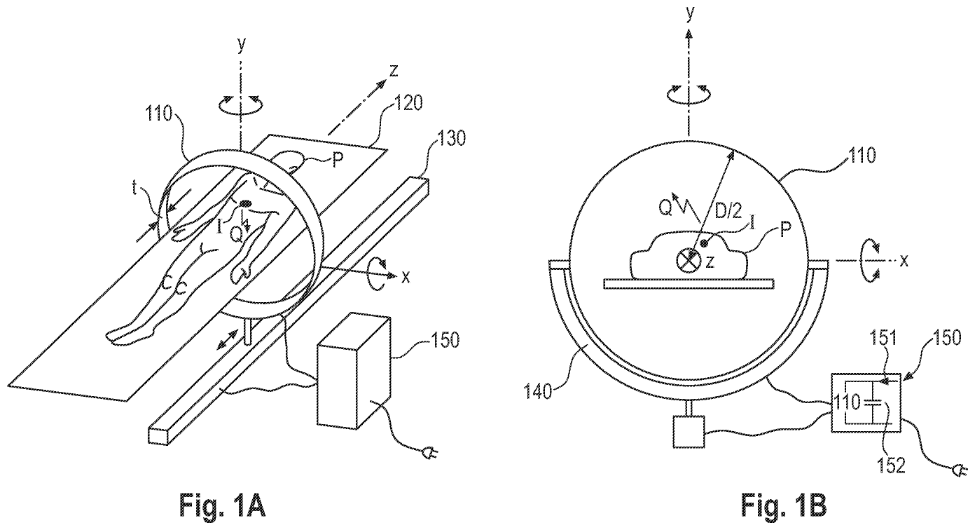

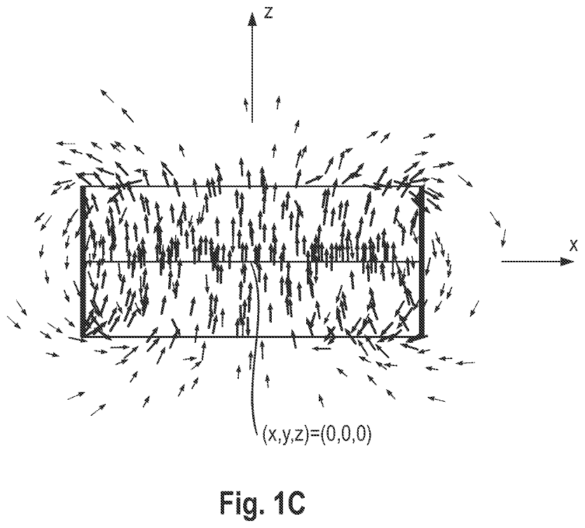

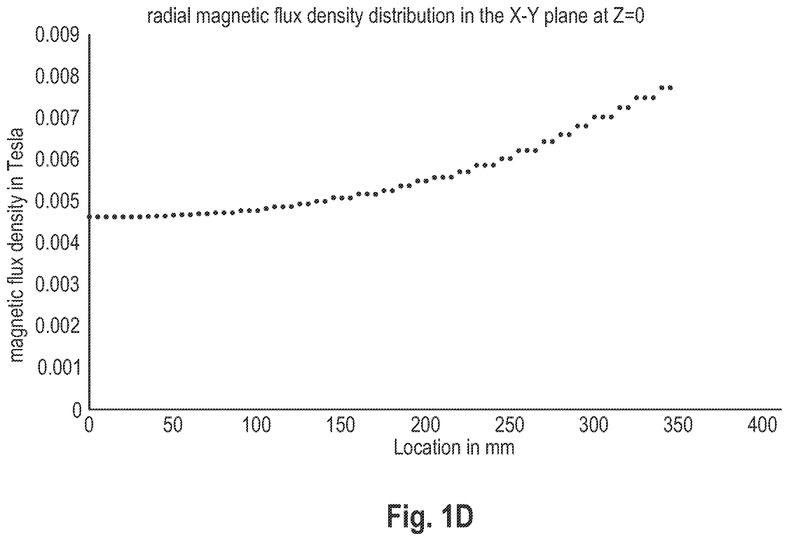

FIGS. 1A and 1B show a first preferred embodiment of the charging device according to the invention, firstly in a perspective view and secondly in a view along a Z-axis shown in the figures, wherein the charging device comprises a coil supported by a gimballed mount.

FIG. 1C shows simulation results of the alternating magnetic field generated by the coil of the charging device of the first preferred embodiment of the invention.

FIG. 1D shows the amplitude of the alternating magnetic field (B-field in mT) in the center of the coil radial to the coil axis.

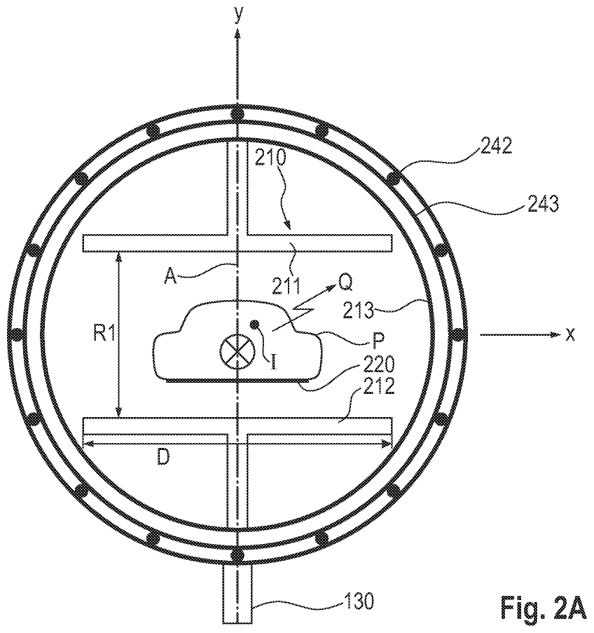

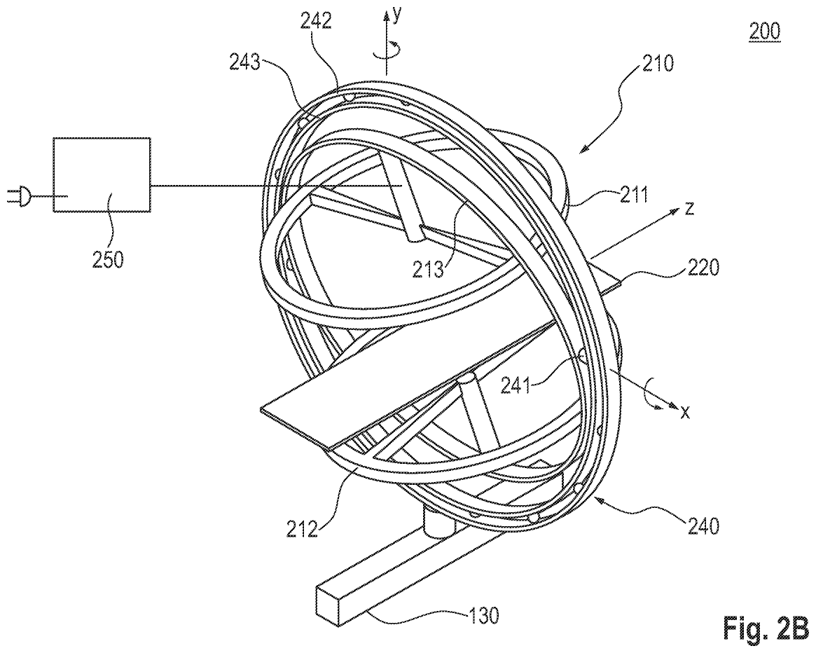

FIGS. 2A and 2B show a second preferred embodiment of the charging device according to the invention, which comprises a coil of a pair of coil formed from two sub-coils, wherein an mount supports both sub-coils and is configured to pivot and/or displace the sub-coils together.

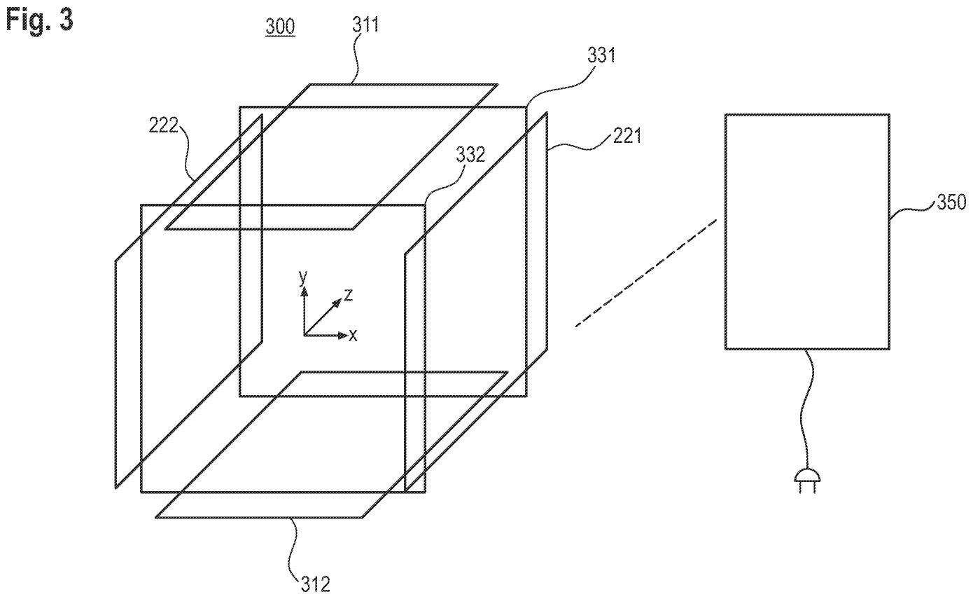

FIG. 3 shows a third preferred embodiment of the charging device according to the invention, which comprises three coils, each of which is configured from two sub-coils, wherein respective coil axes are spatially perpendicular to one another, and, in a superposition area located between the respective coils, the amplitude and the vector of the alternating magnetic field can be adjusted by controlling the coils.

The embodiments of the charging device according to the invention explained below are for charging an energy storage of an implant that is fully implanted in a human or animal body.

The implant constitutes generally an entity that takes over certain functions in the implanted state and, for this purpose, comprises at least an electronics assembly and said energy storage supplying the electronics assembly.

Preferably, the implant can still comprise electrodes for capturing body data (body information) and/or delivering impulses to the body, and preferably a communication unit that acts as an interface to the outside world.

The implant is, for example, a cardiac pacemaker, brain pacemaker, bladder pacemaker, and/or an analysis unit that captures body data, such as blood pressure and/or blood levels.

For the contactless recharging of the energy storage, the implant comprises
i. a charging impulse generation portion with charging electronics, such as those described in patent document DE 10 2019 124 435; and/or
ii. a receiving coil wound around a preferably specially shaped core, the receiving coil with core enabling charging of the energy storage based on the ordinary induction.

An essential element of the charging impulse generation portion is a magnetization portion, for example a Wiegand wire, having oriented magnetic domains which can be influenced by the magnetic field component in its axial direction of an electromagnetic alternating field generated by the embodiments of the charging device according to the invention so that a remagnetization wave in the form of domino-like reversal domains (Weiss domains) runs across the magnetization portion.

The remagnetization wave leads to such a high change in magnetic flux over time that a coil arranged in spatial proximity to the magnetization portion generates a voltage impulse leading to a charging impulse of the energy storage.

This method of recharging the energy storage is particularly advantageous in that the frequency of the alternating magnetic field can be lowered to such an extent that no adverse effects, such as skin effects, prevent the alternating magnetic field from penetrating the body and reaching the magnetization portion.

For example, the magnetizing portion may be a Wiegand wire around which a coil is wound.

The speed of the remagnetization wave running across the Wiegand wire is of the range of 800 m/s in idle operation, wherein the length of the Wiegand wire contained in the pacemaker is in the range of 0.7-1.2 cm. This again results in a running time of the remagnetization wave (wave of reversal domains) in the range of 10-20 has in idle operation.

Taking these values into account, the charging device according to the invention, which will be explained below, generates the alternating magnetic field with a frequency in a range from 0.1 to 10 kHz. In this frequency range, the alternating magnetic field penetrates the very deep-lying areas of the body of the living being and can thus easily reach the magnetization portion of the implant for contactless charging.

Said frequency range is also suitable for charging by means of ordinary induction, at least if said core run through the coil.

The embodiments of the charging device according to the invention are explained below assuming that the implant is a cardiac pacemaker fully implanted in/on the human heart. Nevertheless, the invention is not limited thereto.

The cardiac pacemaker comprises a case that encapsulates the energy storage and the corresponding electronics assembly. The case of the cardiac pacemaker has a volume in the range of 1 cm³.

Necessary electrodes of the cardiac pacemaker are exposed on the surface of the case and contact the portions of the human heart, and/or are formed as anchoring electrodes that are anchored in the human heart and hold the cardiac pacemaker in place.

The cardiac pacemaker receives body data via the electrodes, i.e. information about the activity of the heart, and/or can emit stimulation impulses to the heart via this.

The energy storage of the cardiac pacemaker is, for example, an electrochemical energy storage, in particular an accumulator, for example a lithium-ion accumulator, with such a capacity that it can supply all electronic components of the cardiac pacemaker with electrical energy for a period of between, for example, 0.75 to 1.25 years. If it is recharged every 0.5 years, for example, this ensures that all electronic components are supplied with energy.

Charging is contactless based on ordinary or indirect induction through the charging device described more precisely below.

### FIRST EMBODIMENT

With reference to FIGS. 1A to 1E, a first preferred embodiment of the charging device according to the invention is explained below.

The charging device 100 according to the first preferred embodiment comprises a self-supporting (dimensionally stable) coil 110 that generates the alternating magnetic field necessary for charging. The coil 110 comprises such spatial dimensions that a body support 120 of the charging device 100 intended for a patient P can be accommodated in sections within the coil 110, without coming into contact with the self-supporting coil 110 or its elements such as insulations, frames, windings, etc.

In the preferred embodiment of the charging device 100, the body support 120 is a stretcher on which the patient P lies or is arranged when the charging device 100 is used as intended.

Patient P has a schematically indicated cardiac pacemaker I, which is fully implanted in patient P's heart.

The body support 120 extends through the coil 110 in the direction of a Z-axis shown in FIGS. 1A and 1B. The Z-axis corresponds to a coil axis of the coil 110. When the patient P is lying on the body support 120, as will be described further below, a body axis of the patient is in the direction of the coil axis of the coil 110, and the alternating magnetic field located in the area inside the coil 110 penetrates the body of the patient P and reaches the cardiac pacemaker I for charging the energy storage.

In the preferred embodiment, the coil 110 is a circular coil having a diameter D, with a corresponding coil plane in the X-Y plane shown in FIGS. 1A and 1B perpendicular to the coil axis (Z-axis).

Alternatively, the coil 110 may be a square or rectangular frame coil. The diameter D is 0.72 m in the first preferred embodiment, but may also vary insofar as long as the patient find place inside the coil without coming into contact with it.

For example, the coil 110 has a single-layer winding with a plurality of windings w formed from an electrical wire, for example a copper wire. In the first preferred embodiment, the number of windings is w=10, although this value is merely preferred and may also vary.

The electrical wire forming the winding preferably has a rectangular cross-section of 320 mm², which results from a side length of 32 mm pointing in the direction of the Z axis and a side length of 10 mm running perpendicular to this. In addition, there is an electrical insulation, which covers the surface of the electrical wire, with a thickness of 0.5 mm.

This results in a length I of the coil 110 in the direction of its coil axis (Z axis) of 0.32 m, without taking into account the insulation, or of 0.33 m while taking into account the insulation, wherein for the first preferred embodiment the condition D>I thus applies.

At this configuration of the coil 110, an inductance L of the coil 110 has a value of 87 μH. The invention is not limited by the configuration as explained, and said parameters. In general, it is preferred that the configuration of the coil 110 is such that the inductance is in the following range 0.02 mH<=L<=0.3 mH.

The coil 110 of the charging device 100 according to the invention is preferably operated during charging of the energy storage of the cardiac pacemaker I in such a way that it generates in its interior the alternating magnetic field with an amplitude (magnetic flux density B) in the range of greater than/equal 1 mT, preferably in the range of 2.5 mT<=B<=8.0 mT, in particular in a range of 5 mT.

The alternating magnetic field with these values of magnetic flux density B is located in the area inside the coil 110 and runs along the coil axis of the coil 110.

The magnetization portion contained in the cardiac pacemaker I, which is preferably the Wiegand wire already specified, exhibits, at the values of magnetic flux density B and a frequency f (commutation frequency) of the already specified alternating magnetic field in the range from 0.1 to 10 kHz, in particular at 2 kHz, a reliable behavior in that each commutation of the alternating magnetic field initiates the remagnetization wave with a very high probability and thus leads to the charging impulse of the energy storage.

Also, the energy storage can be charged by ordinary induction in this frequency range, especially at frequencies above 2 kHz, if the receive coil of the cardiac pacemaker has the appropriate core.

The explained configuration of the charging device 100 according to the invention according to the first preferred embodiment is particularly advantageous since electrical operating parameters are in range of magnitude that allow without obstacles the operation of the charging device 100 in a usual medical doctor's practice.

The magnetic flux density B of the alternating magnetic field generated by coil 110 is given by the following relationship:

B=(μ₀*I*w)/D (1)

The charging device 100 according to the invention preferably generates an alternating electric current with a strength of I=300 A in the winding of the coil 110. For said windings w=10, the above relationship (1) results in a magnetic flux density B of 5.25 mT, which is, for reliable triggering of the Wiegand wire/magnetization portion or for sufficient ordinary induction, within the above ranges.

Taking into account the AC resistance of the coil 110 resulting from ω*L, with ω=2πf, an AC voltage necessary for the generation of the AC current I follows from U=I*ω*L and is thus 327 V at a frequency f of 2 kHz of the alternating magnetic field.

Both the value of the alternating current I generated in the winding and the corresponding voltage value U are in ranges of magnitude that can be provided in normal buildings/doctors' practices. Therefore, the charging device 100 according to the invention does not necessarily have to be installed in a separate facility, such as is the case for the operation of an MRT.

Advantageously, this is realized by the coil 110 of the charging device 100 according to the invention being part of a resonant circuit, for example a parallel resonant circuit. In this way, only the effective power (copper losses) has to be taken from the grid.

FIG. 1B schematically illustrates an amplifier 151 and a capacitor 152, which are together contained in a control unit 150 of the charging device 100. Together with the coil 110, the capacitor 152 forms said parallel resonant circuit.

The parallel resonant circuit is sized to resonate when the charging device 100 is operating as intended. Taking into account the inductance of L=87 μH of coil 110, the relationship 1/(ω*C)=ω*L valid for the resonance case results in a capacitance of the capacitor of C=72.8 μF.

The ohmic resistance R of the coil 110 is essentially due to the material and dimensions of the electrical wire forming the winding. In the present first preferred embodiment of the charging device 100 according to the invention, the ohmic resistance R of the electrical wire of the coil 110 is about 1.24 mcg.

This results in an effective power consumption of the coil 110 in the example with a corresponding effective value Jeff of the alternating current flowing through the coil 110 of 56 W. This effective power consumption of the parallel resonant circuit can be easily supplied by a standard power supply in buildings (e.g. 230 V; 50 Hz). That is, the charging device 100 according to the invention can be connected to a standard socket via the control unit 150. FIGS. 1A and 1B show such a connection cable schematically.

When the energy storage of the cardiac pacemaker I needs to be recharged in the implanted state, the patient P lies down on the stretcher 120 as shown in FIGS. 1A and 1B.

A spatial position of the patient P or the stretcher 120 relative to the coil 110 with respect to the Z-direction (positive or negative direction of the Z-axis) is achieved by the control unit 150 of the charging device 100 displacing an mount 140 of the coil 110, which will be explained below, relative to the stretcher 120/patient P. The mount supports the dimensionally stable coil 110 such that it is self-supporting. For this purpose, the charging device 100 has a linear guide 130 which is arranged below the stretcher 120 and which holds the mount 140 of the coil 110 displaceable in Z-direction.

The controller 150 is configured to control the linear guide 130 to move the mount 140 to a specific position in the Z-direction. Alternatively/additionally, the body support/stretcher 120 may be displaceably supported so that the control unit 150 of the charging device 100 may displace the body support/stretcher 120 by controlling displacement mechanisms, to contactlessly position the patient P relative to the coil 110.

The patient P and the coil 110, when charged as intended, have such a spatial relationship to each other that the cardiac pacemaker I is located in the area along the coil axis within the coil 110.

This condition is given in FIGS. 1A and 1B in that the cardiac pacemaker I is located in the cylindrical volume V defined by the coil 110, which is given by (D/2)²*π*I. The alternating magnetic field running along the coil axis in this area penetrates the body of the patient P and reaches the cardiac pacemaker I.

The magnetic flux density B has adequate values for charging in the entire cylinder volume, which is why the specific position of the cardiac pacemaker I within the cylinder volume V plays a subordinate role as long as the orientation of the magnetization portion or the core serving for ordinary induction is correct. This can be seen from the following explanations of FIGS. 1C to 1E.

FIGS. 1C and 1D show the magnitude and orientation of the magnetic flux density B of the alternating magnetic field generated by coil 110 when the alternating current of I=300 A flows through the winding of coil 110. FIG. 1C corresponds to a sectional view of the coil 110, wherein the sectional plane corresponds to the Z-X plane shown in FIGS. 1A and 1B and includes the center of the coil 110. The center of coil 110 is also the origin of the coordinate system shown in FIGS. 1A and 1B.

In this context, FIG. 1D shows the amplitude of the magnetic flux density B and FIG. 1C the corresponding vector representation, wherein the figures relate to the values resulting in space accordingly.

FIG. 1D indicates clearly that the amplitude of the magnetic flux density B within the coil 110, particularly in the mid-plane area (X-Y plane, with Z=0), has an amplitude greater than 4.5 mT throughout the diameter D (see areas in FIG. 1D). In addition, as FIG. 1C illustrates, the orientation (vector) of the magnetic flux density B in the mid-plane area is essentially homogeneous, i.e. the vectors indicating the orientation of the magnetic flux density B are parallel to the coil axis.

FIG. 1D shows in detail how the amplitude of the resulting magnetic flux density B in the center plane of coil 110 (Z=0) changes in the radial direction (X and/or Y direction) starting from the coil axis or the origin of the coordinate system. The magnetic flux density B has a resultant magnetic flux density of 4.5 mT at (X, Y, Z)=(0, 0, 0) and increases in the radial direction to about 8 mT.

According to its intended use, the charging device 100 of the first preferred embodiment is operated in such a way that the cardiac pacemaker I is located in the cylinder volume V defined by the coil 110 when the corresponding energy storage is charged. However, it is particularly preferred that the patient P be positioned relative to the coil 110 when the charging device 100 of the invention is operating as intended, such that the cardiac pacemaker I is located at a point in the mid (X-/Y-)-plane of the coil 110.

The magnetization portion, in particular the Wiegand wire, or the core of the cardiac pacemaker I is oriented during the intended operation of the charging device 100 in such a way that the running direction, as intended, of the remagnetization wave or a longitudinal extension of the core coincides with the direction of the coil axis/Z-axis and thus with the main component (Z-component) of the resulting magnetic flux density B. The running direction, as intended, corresponds to the direction in which the remagnetization wave is to run across the magnetization portion in order to achieve optimal voltage impulses or charging impulses. For said Wiegand wire, this running direction usually corresponds to its longitudinal axis.

The alignment of the magnetization portion/running direction of the remagnetization wave or longitudinal extension of the core serving for ordinary induction with the direction of the coil axis is achieved by paying attention to the orientation when implanting the cardiac pacemaker I or the magnetization portion contained therein. For example, the cardiac pacemaker I has electrodes and or anchorages formed in such a way that the orientation of the magnetization portion/longitudinal extension of the core serving for the ordinary induction in the direction of the body axis (and thus in Z-axis/coil axis) results inevitably at the time of implantation.

The orientation of the magnetization portion, in particular the orientation of the longitudinal extension of the Wiegand wire, or the longitudinal extension of the core serving for the ordinary induction in the direction of the Z-axis/coil axis, means that any commutation of the alternating electromagnetic field reliably initiates the remagnetization wave/ordinary induction, and a desired voltage impulse occurs, which in turn leads to the charging impulse for the energy storage.

The explanations of the alternating magnetic field generated by the coil 110 indicate that the reliable initiation of the remagnetization wave or the reliable induction is independent of where the cardiac pacemaker or the magnetization portion is located in the cylinder volume V defined by the coil 110. This is because there is a sufficiently homogeneous amplitude of the magnetic flux density B within the coil 110 with simultaneous orientation of the B vector pointing in the coil axis (independent of location).

This eliminates the need for meticulous adjustment and adaptation of the strength and orientation of the alternating magnetic field to the position and location of the cardiac pacemaker.

Even if the cardiac pacemaker I has been implanted in the body of the patient P in such a way that the direction of travel of the remagnetization wave or the longitudinal extension of the core points in the direction of the body axis of the patient, deviations may occur between the longitudinal extension of the core or the orientation of the magnetization portion and thus the running direction on the one hand and the coil axis on the other hand, during operation of the charging device 100, as intended.

This can be attributed to individual physiological conditions of the patient, which lead to a deviating position of the cardiac pacemaker I in the patient's body. On the other hand, simple physical movements of the patient during the charging process can lead to deviations.

In order to bring and/or maintain the charging of the energy storage in an optimal condition, the mount 140 is configured to be adjustable.

In one aspect, the coil 110 can be displaced in the direction of the Z-axis or coil axis by the linear guide 130.

On the other hand, the mount 140 is configured as a gimballed mount. This allows the coil 110 to pivot about the respective X-axis and Y-axis shown in FIGS. 1A and 1B.

The control unit 150 is configured to use said adjustment options in order to optimize the charging of the energy storage.

The optimization of charging requires a conclusion regarding the extent to which the commutation of the alternating magnetic field results in the desired, sufficiently high charging impulses. If the orientation of the intended running direction of the remagnetization wave or the longitudinal extension of the core deviates too much from the coil axis, the amplitude of the voltage impulses generated is reduced to such an extent that sufficient charging pulses for the energy storage can no longer be achieved.

The cardiac pacemaker I preferably generates a quality signal Q as feedback, which reflects the efficiency of the charging. This quality signal can be sent actively or requested passively.

The quality signal Q sent can, for example, be a low-frequency signal that penetrates the body of the patient P and, if no external antenna is provided, the sleeve or case of the cardiac pacemaker.

The quality signal can be requested, for example, by the cardiac pacemaker dampening defined frequencies.

The quality of the charging impulse is proportional to the value of the integral (∫idt). At good charging impulses, i.e. with very high quality, the value amounts up to 1000 nC, for example. The signal Q indicating the quality can, for example, indicate the value of the integral of the current of the charging impulse over time (∫idt), i.e. its charge content.

The electronics assembly of the cardiac pacemaker I can be configured in such a way that it transmits the signal Q indicating the quality for each charging impulse or, alternatively, only for those charging impulses that occur at certain intervals one after the other. The intervals are, for example, 25, 50, 100, 200, 500, 750, or 1000 charging impulses. It is also possible to send a value corresponding to the average value of all charging impulses that occurred in an interval. In this way, the energy required for active transmission of the data can be reduced.

The control unit 150 comprises a receiving unit and is configured to evaluate the quality signal Q from the cardiac pacemaker I. Based on the quality signal Q, the control unit 150 performs appropriate steps to optimize charging by pivoting the coil 110 about the X-/Y-axes accordingly and/or displacing it in the Z direction until the quality signal indicates optimal orientation.

Preferably, the quality signal Q is proportional to the efficiency of the charging.

Alternatively, the signal S indicating the quality of the charging impulse may be a binary signal, for example, which assumes an OK state when the charging impulse or the charge content thereof exceeds a threshold and an NG state when the charging impulse does not exceed the threshold. For example, the threshold may be 50%, 55%, 60%, 65%, 70%, 75%, 80%, 85%, or 90% of the charge content deliverable by the magnetization portion.

If the quality signal Q is a binary signal, the threshold value for the OK state of the signal is preferably in higher ranges, because the threshold value serves as a whole as a criterion for terminating the pivoting of the coil 110 about the respective axes and for evaluating the achieved position of the coil as optimal. For example, the extreme positions (change from good to bad or vice versa) are here evaluated.

In general, the charging device 100 according to the invention can also be used with implants that do not emit a quality signal Q, but only have the functionality that the control unit 150 can request the state of charge of the energy storage. For the application shown, for example, the corresponding electronics assembly of the cardiac pacemaker I can be configured in such a way that the charge state of the corresponding energy storage can be queried via a transmit and receive function of the cardiac pacemaker I.

Thus, as an alternative to the signal Q indicating the quality, the control unit 150 can obtain the conclusion on the efficiency of charging from a repeated query of the state of charge of the energy storage during charging. In detail, the control unit 150 can draw conclusion about the quality/efficiency of the individual charging impulses by information about a change in the state of charge, the time interval of the queries, a known maximum level of the charging impulses, and the commutation frequency. The time interval at which the control unit 150 requests the state of charge of the energy storage is preferably 0.5 min, 1.0 min, 1.5 min, 2.0 min, 2.5 min, 3.0 min, 3.5 min, 4.0 min, 4.5 min, 5.0 min.

When the control unit 150 has gained knowledge of the quality of the charging impulses, respectively, it controls the gimballed mount 140 to pivot the coil 110 about the respective axes (X-axis and/or Y-axis) and/or displace it along the Z-axis to optimize charging. The pivoting and/or displacement takes place in such a way that the patient does not come into contact with the self-supporting (dimensionally stable) coil or the elements thereof (contactless).

### SECOND EMBODIMENT

In the following, a second preferred embodiment of the invention is explained with reference to FIGS. 2A and 2B.

FIG. 2A shows the configuration of a charging device 200 according to the second preferred embodiment of the invention as seen in the direction of the Z-axis, and FIG. 2B shows a perspective view.

The charging device 200 is used to charge an energy storage of a cardiac pacemaker I, which has an identical configuration to that described with reference to the first embodiment. In this respect, reference is made to the explanations on the first embodiment with regard to the configuration and functionality of the cardiac pacemaker I.

The charging device 200 comprises a self-supporting coil 210 configured to generate the alternating magnetic field required for indirect charging of the energy storage. In FIG. 2A, the coil axis A of coil 210 runs in the direction of the Y axis of the coordinate system shown there.

In contrast to the first preferred embodiment, the winding of the coil 210 is not continuous but preferably pulled apart in the center of the coil 210, which at the same time corresponds to the origin of the coordinate system, in such a way that the coil 210 is formed of sub-coils.

The coil comprises two circular sub-coils 211 and 212, which are axially arranged with respect to each other and have the same diameter D. The sub-coils 211 and 212 are arranged at a distance R1 from each other and have the same sense of winding.

The winding of coil 210 extends over a connecting arm 213, connecting parts of the winding that form sub-coils 211 and 212. In this embodiment, the connecting arm 213 is a closed ring that is preferably circular.

The winding is single-layered, as in the first preferred embodiment, and the portions of the winding forming the sub-coils necessarily have the same sense of winding.

In the present embodiment, the electrical wire forming the sub-coils 211, 212 preferably has the same cross-sectional dimensions as those in the first preferred embodiment; rectangular with 32 mm side length in the direction of the coil axis and 10 mm side length in the direction radial to the coil axis. A portion of the electrical wire that run across the connecting arm 213 may have the above cross-sectional dimensions or may be sized differently.

For example, each of the sub-coils 211 and 212 has a diameter of 160 cm and has five windings (w=5). The distance R1 between the sub-coils 211 and 212 is preferably equal to the radius of the sub-coils, i.e. 80 cm in the present embodiment, or is preferably smaller than the radius. The diameter D of the sub-coils 211 and 212 can be made larger if a larger distance R1 is required between the sub-coils.

A mount 240 supports the dimensionally stable coil 210 so that it is self-supporting, by adjustably supporting the ring 213 forming the connecting arm 213, the mount 240 having adjustment mechanisms for this purpose.

The adjustment mechanisms comprise a pivot bearing 241 that allows the connecting arm 213, and thus the coil 210, to pivot or rotate about the X axis of the coordinate system shown in FIGS. 2A and 2B. In addition, the adjustment mechanisms have ball bearings 242 that allow the connecting arm/ring 213, which is connected to a bearing ring 243 via the pivot bearing 241, to pivot or rotate overall about the Z-axis shown in FIGS. 2A and 2B. As will be understood from this, the connecting arm/ring 213 can be rotated by the ball bearings 242 supporting the bearing ring 243 to such an extent (90 degrees) that the rotational bearing 241 does not correspond to the X-axis shown in FIGS. 2A and 2B, but to the Y-axis shown.

The adjustment mechanisms (pivot bearing 241, ball bearing 242) allow the coil 210 to be aligned in a desired orientation relative to the implanted cardiac pacemaker I.

Additionally, the charging device 200 of the second preferred embodiment of the invention may comprise the linear guide 130 already discussed with reference to the first preferred embodiment. Reference is made to the corresponding explanations in the context of the first preferred embodiment. As in the first preferred embodiment, the stretcher 220 may be preferably displaceable in the Z direction. If this is the case, the linear guide 130 is preferably not present.

A control unit 250, configured to perform the functions explained with reference to the first preferred embodiment, is electrically connected to both the mount 240 and the coil 210. As in the first preferred embodiment, the coil 210 is part of a parallel resonant circuit not shown in FIG. 2A, with the corresponding other elements of the resonant circuit, such as said amplifier and capacitor, being contained in the control unit 250.

A body support 220, which is a stretcher as in the first preferred embodiment, extends between sub-coils 211 and 212 perpendicular to the drawing plane of FIG. 2A in the direction of the Z axis.

When the energy storage of the cardiac pacemaker I of the patient P needs to be charged, the patient P lies down on the stretcher 220 and is located between the sub-coils 211 and 212. Like the stretcher 220, the body axis or longitudinal axis of the patient P extends in the direction of the Z axis shown. Patient P is shown schematically in FIG. 2A.

Coil 210 generates a uniform electromagnetic alternating field even though the winding is not continuous, but is pulled apart in the form of sub-coils 211 and 212. In other words, due to the configurations, dimensions and spatial relation to each other explained in the foregoing, both sub-coils 211, 212 act as a single coil with continuous winding. In the present second embodiment, the coil 210 preferably has the configuration of a so-called Helmholtz coil.

Both sub-coils 211 and 212 each generates a portion of the total alternating magnetic field generated, with the magnetic field extending within coil 210 along coil axis A running across interior spaces of sub-coils 211, 212 and an area between sub-coils 211, 212.

The area between the sub-coils is a superposition area in which the parts of the alternating magnetic field generated by the sub-coils are superimposed in such a way that the resulting alternating magnetic field extends along the coil axis A.

The vectors indicating the orientation of the alternating magnetic field passing through the interiors of the sub-coils 211, 212 and superposition area located between the sub-coils show parallel courses. Moreover, under the same assumptions/operation parameters as shown with the first embodiment, the magnetic flux density B in the superposition area has values exceeding 4 mT over long distances.

These values are sufficient to initiate the explained remagnetization wave that runs across the magnetization portion or Wiegand wire of the cardiac pacemaker I and leads to the charging pulse of the energy storage, or to initiate the ordinary induction leading to the charging impulse.

In contrast to the first preferred embodiment, the alternating magnetic field located within the coil along the coil axis A does not penetrate the body of the patient P parallel to its body axis running in the Z direction, but perpendicular to it.

The charging device 200 is therefore intended in particular for the case where the cardiac pacemaker I is oriented in the body of the patient P in such a way that the intended running direction of the remagnetization wave of the magnetization portion or the longitudinal extension of the core does not point in the direction of the body axis (Z direction), but deviates significantly therefrom and runs essentially in the Y and/or X direction.

Adjustment of an optimal position and alignment of the coil 210 with respect to the patient's body P proceeds similarly to the first preferred embodiment of the charging device according to the invention in that the control unit 250 controls the adjustment mechanisms and/or the linear guide 130 and adjusts and orients the coil 210 relative and contactless to the patient/cardiac pacemaker I until optimal charging occurs.

### THIRD EMBODIMENT

FIG. 3 shows a third preferred embodiment of a charging device 300 according to the invention. The charging device 300 is also used to charge an energy storage of an implant I, such as the cardiac pacemaker I discussed previously.

With respect to the structure and functionalities of the cardiac pacemaker I, reference is made to the explanations of the first and second embodiments.

The charging device 300 generates the alternating magnetic field necessary to charge the energy storage in a manner similar to the coil 210 of the charging device 200 according to the second preferred embodiment, but eliminates the need for a mount to change the spatial orientation of the alternating magnetic field.

For this purpose, the charging device 300 has a first coil, a second coil, and a third coil, which are arranged in a self-supporting and dimensionally stable manner.

The first coil comprises a first coil pair formed from a first sub-coil 311 and a second sub-coil 312, the corresponding coil axis A being in the direction of or corresponding to the Y-axis of the corresponding coordinate system shown in FIG. 3.

The second coil comprises a second coil pair equally configured from a first sub-coil 221 and a second sub-coil 222. The coil axis B of the second coil corresponds to the X axis of the corresponding coordinate system shown in FIG. 3.

Ultimately, the charging device 300 comprises a third coil, which in turn is configured from a first sub-coil 331 and a second sub-coil 332. The coil axis C of the third coil corresponds to the Z axis of the coordinate system shown in FIG. 3.

All coil axes are preferably spatially perpendicular to each other.

In contrast to the preceding embodiment, the coils or sub-coils are not circular, but are configured as frame coils with rectangular coil planes.

All sub-coils, which respectively form the coil pairs of the coils, preferably have the same structure with regard to the electrical conduction and number of windings w, forming the corresponding winding and have, respectively, the same sense of winding. Dimensions of the electrical wire and number of windings w are preferably identical to those of the first and second preferred embodiments. The first, second and third coils are preferably connected to one another in such a way that they mutually support each other and are dimensionally stable, self-supporting in space.

A distance between the sub-coils 221, 222 in the X-direction is dimensioned in relation to their dimensions perpendicular to the coil axis A, i.e. in planes parallel to the Y-Z plane shown in FIG. 3, so that the sub-coils 221, 222 act as a single coil (Helmholtz coil). This condition preferably applies to all sub-coils shown in FIG. 3.

The charging device 300 of the third preferred embodiment comprises a control unit 350 configured to separately drive each coils and generate an alternating current in the respective windings. Each coil and the corresponding sub-coils, respectively, thus generates an individual alternating magnetic field.

For this purpose, as in the preceding embodiments, each of the coils is part of a separate oscillating circuit whose elements are contained in the control unit 350. With respect to the strength of the alternating current generated in each coil and the structure of the oscillating circuits, reference is made to the explanations on the preceding embodiments.

When the control unit 350 generates the alternating current in the pairs of coils/sub-coils 311 and 312, the individual alternating magnetic field located within the coil along the coil axis A passes through the subcoils 311, 312 and over the superposition area located between the subcoils.

The sub-coils 311 and 312 ergo generate the alternating magnetic field in the same manner as the coil 210 in the second preferred embodiment. These explanations apply to all sub-coils of the corresponding coils shown in FIG. 3.

The individual alternating magnetic fields of all coils superimpose in a common area to form the alternating magnetic field that reaches implant I.

The alternating magnetic field reaching the cardiac pacemaker I has sufficiently high and homogenous values in the common area over wide spatial areas around the coordinate origin to trigger the magnetization portion (Wiegand wire) of the cardiac pacemaker I to generate the charging impulse or to flow through the core serving for ordinary induction to generate the charging impulse.

The orientation of the resulting alternating magnetic field can be changed by the control unit 350 by driving the respective coils differently and varying the alternating current flowing in the corresponding windings. If the orientation of the resulting alternating magnetic field should not be necessary in space, but only in one plane, one of the three coils is superfluous.

When the charging device 300 is used as intended, the patient P is preferably pushed in the direction of the Z-axis through the partial coils 331, 332 to such an extent that the upper body of the patient P and the cardiac pacemaker I is located in the common area. Too, in this embodiment, the movement of the patient takes place without coming into contact with all coils and its elements, respectively.

Here, as in the other embodiments, the patient P may be arranged, for example, on a body support or stretcher.

Common to all the embodiments of the charging device according to the invention explained hereinabove is that the alternating magnetic field located within the coil(s) along the corresponding coil axis is used to charge the energy storage. The alternating magnetic field or its magnetic flux density B is so strong and largely homogeneous over long distances in these areas of the coil(s) that precise knowledge of the position of the implant (pacemaker) is of secondary importance if the direction of the axis of the magnetization portion or the longitudinal extension of the core coincides with the axis of the magnetic field vector to a first approximation.

In addition, all embodiments allow the vector of the alternating magnetic field to pivot/rotate without changing the amplitude of the magnetic flux density B. This is done by mechanically pivoting/rotating the coil(s) and/or by changing the superposing magnetic alternating fields without contact with the patient. This allows an extremely reliable orientation of the magnetic field vector in the direction of the longitudinal axis of the core or the axis of the magnetization portion and thus in the direction of travel of the remagnetization wave.

1. A charging device (100, 200, 300) for contactlessly charging an energy storage of an implant (I) implanted in a body of a living being, comprising:
at least one self-supporting coil (110, 210) extending along a coil axis and configured to generate alternating magnetic field; wherein
during intended use of the charging device (100, 200, 300), the body is arranged relative to the coil (110, 210) so that the alternating magnetic field extending in the area inside the coil along the coil axis penetrates the body for charging the energy storage.

2. The charging device (100, 200, 300) of claim 1, wherein the charging device is configured to rotate a vector of the alternating magnetic field for charging at least two-dimensionally.

3. The charging device (100, 200, 300) according to claim 1, wherein the charging device is configured to rotate the vector of the alternating magnetic field for charging without changing the corresponding amplitude.

4. The charging device (100, 200, 300) according to the patent claim 1, wherein
during the intended use of the charging device, the body is arranged relative to the self-supporting coil in such a way that a longitudinal axis of the body runs in the direction of the coil axis and is located within the coil, wherein the alternating magnetic field reaches the location of the implant.

5. The charging device (100, 200, 300) according to the patent claim 2 further comprising:
an mount which supports the self-supporting coil and is configured to pivot the coil relative to the body around at least one, preferably two axes, to rotate the vector; wherein
the charging device is configured to control the mount in order to pivot the coil to a certain orientation relative to the body for optimizing the charging of the energy storage.

6. The charging device (100, 200, 300) according to the patent claim 5, wherein
the mount supporting the coil is displaceable relative to the body and/or a body support for supporting the body is displaceable relative to the coil; and wherein
the charging device is adapted to displace the mount and/or the body support to bring the coil into a specific position relative to the body for optimizing the charging of the energy storage.

7. The charging device (100, 200, 300) according to the patent claim 5, wherein
the charging device comprises a receive unit configured to either receive or request a quality signal generated in the implant reflecting the efficiency of the charging; and
the charging device is configured to pivot and/or move the coil to the specific orientation and/or position as a function of the quality signal to optimize charging.

8. The charging device (100, 200, 300) according to claim 1, wherein
a magnetic flux density of the alternating magnetic field along the coil axis has a value B, wherein
1.0 mT<=B<=20.0 mT,
Preferably
2.0 mT<=B<=20.0 mT,
2.5 mT<=B<=8.0 mT,
3.5 mT<=B<=7.0 mT,
4.5 mT<=B<=6.0 mT,
4.8 mT<=B<=5.2 mT, or
5.0 mT=B.

9. The charging device (100, 200, 300) according to claim 1, wherein the coil is formed from two pulled apart sub-coils arranged at a distance R1 from one another on the same axis, which cooperate in such a way that the alternating magnetic field passes, along the coil axis, through the sub-coils and an area located between the sub-coils; wherein preferably R1 is equal to D/2 (Helmholtz coil) and,
during intended use of the charging device, the body is arranged relative to the sub coils in such a way that the, preferably mainly homogenous, alternating magnetic field located between the subcoils penetrates the body for charging the energy storage.

10. The charging device (100, 200, 300) according to the patent claim 9, further comprising:
an mount which supports the two sub coils of the first coil and is configured to pivot the first coil relative to the body around at least one, preferably two axes, to rotate the vector; and wherein
the charging device is configured to control the mount in order to pivot the two sub coils of the first coil to a certain orientation relative to the body for optimizing the charging of the energy storage.

11. The charging device (100, 200, 300) according to the patent claim 9, wherein
the charging device is adapted to pivot the two sub coils of the first coil to the specific orientation relative to the body for optimizing charging of the energy storage in response to a quality signal emitted by the implant reflecting the efficiency of the charging.

12. The charging device (100, 200, 300) according to the patent claim 8, further comprising:
a second coil which extends along a coil axis and which is formed from two sub-coils which are arranged at a distance R2 from one another in such a way that an area is located between the sub-coils of the second coil, wherein
the coil axes of the first and second coils are transverse, preferably perpendicular, to each other, such that the magnetic fields between the sub coils of the first and second coil superpose in a common area, and
the charging device is configured to control the first coil and the second coil for optimizing the charging of the energy storage in such a way that a direction of the vector of the alternating magnetic field in the superposition area rotates two-dimensionally (in a plane).

13. The charging device (100, 200, 300) according to the patent claim 12, further comprising:
a third coil which extends along a coil axis and which is formed from two sub-coils, which are arranged at a distance R3 from one another in such a way that an area is located between the sub-coils of the third coil, wherein
the coil axes of the first, second and third coils run transversely, preferably along spatial coordinates X, Y and Z to each other such that the magnetic fields between the sub coils of the first, second and third coil superpose in the common area; and
the charging device is configured to control the first coil, the second coil and the third coil for optimizing the charging of the energy storage in such a way that a direction of the vector of the alternating magnetic field in the superposition area rotates three-dimensionally.

14. The charging device (100, 200, 300) according to the patent claim 11, wherein
the charging device is configured to either receive or request a quality signal generated in the implant reflecting the efficiency of the charging; and
the charging device is configured to bring the vector of the alternating magnetic field in the superposition area into a defined position in order to optimize the charging of the energy storage.
